(19) United States
(12) Patent Application Publication
(10) Pub. No.: US 2004/0229791 A1
(43) Pub. Date: Nov. 18, 2004

(54) Compositions and methods of modulating TGF-beta activity by fatty acids

(76) Inventor: Jung San Huang, St. Louis, MO (US)

Correspondence Address: SAINT LOUIS UNIVERSITY, OFFICE OF RESEARCH ADMINISTRATION, 3556 CAROLINE STREET, ST. LOUIS, MO, 63104, US

(21) Appl. No.: 10/748,703

(22) Filed: Dec. 30, 2003

Related U.S. Application Data
(60) Provisional application No. 60/437,034, filed on Dec. 31, 2002.

(51) Int. Cl. A61K 38/18; A61K 31/20; A61K 31/202

(52) U.S. Cl. 514/12; 514/558; 514/560

(57) ABSTRACT

The present invention comprises compositions and methods for modulating or augmenting growth factor activity, especially TGF-β activity, by administering a fatty acid. The invention is based upon the discovery that fatty acids, especially those fatty acids having a carbon skeleton of at least 14 carbons, bind to α2-macroglobulin, prevent binding of TGF-β to α2-macroglobulin, and disrupt TGF-β-α2-macroglobulin complexes, which results in an effective increase in TGF-βivity. Fatty acids that bind to α2-macroglobulin are useful in therapies for diseases that involve TGF-β or other growth factors, which are regulated by α2-macroglobulin binding.

### PARENT CASE TEXT

This application claims benefit of priority to U.S. Provisional Patent Application No. 60/437,034, which was filed on Dec. 31, 2002.

### GOVERNMENT SUPPORT CLAUSE

This work was supported by National Institutes of Health Grant CA 38808. The United States Government has certain rights to this invention.

### BACKGROUND OF THE INVENTION

1. Field of the Invention

The present invention relates to the modulation of growth factor activity, especially TGF-β, by the administration of fatty acids, which bind to (α2-macroglobulin, thereby blocking TGF-β-α2-macroglobulin complex formation or disrupting preformed TGF-β-α2-macroglobulin complexes. Fatty acids may be administered to a patient suffering from a disease mediated by or affected by low levels of TGF-β.

2. Description of the Related Art

References, which are listed below, are cited throughout this application by their respective numerical assignments. All references cited in this specification are hereby incorporated by reference. The discussion of references herein is intended merely to summarize the assertions made by their authors and no admission is made that any reference constitutes prior art. Applicants reserve the right to challenge the accuracy and pertinence of the cited references.

1. Roberts, A. B., and Sporn, M. B. (1990) in Handbook of Experimental Pharmacology: Peptide Growth Factors and Their Receptors (Sporn, M. B., and Roberts, A. B., eds) Vol. 95, Part 1, pp. 419-472. Springer-Verlag. New York.

2. Massagué J. (1990) Ann. Rev. Cell Biol. 6, 597-641.

3. Gaedeke, J., Peters, H., Noble, N. A., and Border, W. A. (2001) Contribu. Nephrol. 135, 153-160.

4. O'Kane, S., and Ferguson, M. W. (1997) Intl. J. Biochem. Cell Biol. 29, 63-78.

5. Jennings, J. C., Mohan, S., Linkhart, T. A., Widstrom, R., and Baylink, D. J. (1988) J. Cell. Physiol. 137, 167-172.

6. Merwin, J. R., Newman, W., Beall, L. D., Tucker, A., and Madri, J. (1991) Am. J. Pathol. 138, 37-51.

7. Qian, S. W., Burmester, J. K., Sun, P. D., Huang, A., Ohlsen, D. J., Suardet, L., Flanders, K. C., Davies, D., Roberts, A. B., and Sporn, M. B. (1994) Biochem. 33, 12298-12304.

8. Shah, M., Foreman, D. M. and Ferguson, M. W. (1995) J. Cell Sci. 108, 985-1002.

9. Lyon, M., Rushton, G., and Gallagher, J. T. (1997) J. Biol. Chem. 272, 18000-18006.

10. Rotzer, D., Roth, M., Lutz, M., Lindemann, D., Sebald, W., and Knaus, P.(2001) EMBO J. 20, 480-490.

11. O'Connor-McCourt, M. D., and Wakefield, L. M. (1987) J. Biol. Chem. 262, 14090-14099.

12. Huang, S. S., O'Grady, P., and Huang, J. S. (1988) J. Biol. Chem. 263, 1535-1541.

13. Danielpour, D., and Sporn, M. B. (1990) J. Biol. Chem. 265, 6973-6977.

14. Crookston, K. P., Webb D. J.,Wolf, B. B., and Gonias, S. L. (1994) J. Biol. Chem. 269, 1533-1540.

15 Philip, A., and O'Connor-McCourt, M. D. (1991) J. Biol. Chem. 266, 22290-22296.

16. James, K. (1990) Immunol. Today. 11, 163-166.

17. Borth, W. (1992) FASEB J. 6, 3345-3353.

18. Feige, J. J., Negoescu, A., Keramidas, M., Souchelnitsky, S., and Chambaz, E. M. (1996) Horm. Res. 45, 227-232.

19. Liu, Q., Ling. T.-Y., Shieh, H.-S., Johnson, F. E., Huang, J. S., and Huang, S. S. (2001) J. Biol. Chem. 276, 46212-46218.

20. Salvesen, G., and Enghild, J. J. (1993) Meth. Enzymol. 223, 121-141.

21. Kurecki, T., Kress, L. F., and Laskowski, M., Sr. (1979) Analyt. Biochem. 99, 415-420.

22. Hall, S. W., Humphries, J. E., and Gonias, S. L. (1991) J. Biol. Chem. 266, 12329-12336.

23. Huang, S. S., Zhou, M., Johnson, F. E., Shieh, H. S., and Huang, J. S. (1999) J. Biol. Chem. 274, 22754-22758.

24. Chen, C. A., and Okayama, H. (1988) Biotechniques. 6, 632-638.

25. Piper, H. M., and Das, A. (1986) Basic Res. Cardiol. 81, 373-383.

26. Starnes, H. F., Jr., Warren, R. S., Jeevanandam, M., Gabrilove, J. L., Larchian, W., Oettgen, H. F., and Brennan, M. F. (1988) J. Clin. Invest. 82, 1321-1325.

27. Richieri, G. V., Ogata, R. T., and Kleinfeld, A. M. (1992) J. Biol. Chem. 267, 23495-23501.

28. Wilson, D. M., and Binder, L. I. (1997) Am. J. Path. 150, 2181-2195.

29. Wrana, J. L. Attisano, L., Carcamo, J., Zentella, A., Doody, J., Laiho, M., Wang, X. F., Massague, J. (1992) Cell. 71, 1003-1014.

30. Strickland, D. K., Ashcom, J. D., Williams, S., Burgess, W. H., Migliorini, M., and Argraves, W. S. (1990) J. Biol. Chem. 265, 17401-17404.

31. Cheifetz, S., Andres, J. L., and Massague, J. (1988) J. Biol. Chem. 263, 6984-6991.

32. Border, W. A., Noble, N. A., Yamamoto, T., Harper, J. R., Yamaguchi, Y., Pierschbacher, M. D., and Ruoslahti, E. (1992) Nature. 360, 361-364.

33. Murphy-Ullrich, J. E., Schultz-Cherry, S., and Hook, M. (1992) Mol. Biol. Cell. 3, 181-188.

34. Demetriou, M., Binkert, C., Sukhu, B., Tenenbaum, H. C., and Dennis, J. W. (1996) J. Biol. Chem. 271, 12755-12761.

35. Taipale, J., Miyazono. K., Heldin, C. H., and Keski-Oja, J. (1994) J. Cell Biol. 124, 171-181.

36. Flaumenhaft, R., Abe, M., Sato, Y., Miyazono, K., Harpel, J., Heldin, C. H., and Rifkin, D. B. (1990) J. Cell Biol. 120, 995-1002.

37. Webb, D. J., Roadcap, D. W., Dhakephalkar, A., and Gonias, S. L. (2000) Prot. Sci. 9, 1986-1992.

38. Bruning, P. F., and Bonf□er, J. M. G. (1986) Canc. Res. 46, 2606-2609.

39. Mendel, C. M., Frost, P. H., and Cavalieri, R. R. (1986) Clin. Endocrinol. Metab. 63, 1394-1399.

Transforming growth factor β (TGF [symbol] -) is a family of 25-kDa structurally homologous dimeric proteins, which show approximately 70% amino acid sequence homology (1,2). It has a remarkably wide range of activities. It inhibits growth of epithelial cells, endothelial cells and lymphocytes, but stimulates growth of mesenchymal cells such as fibroblasts. It has chemotactic activity toward mesenchymal and inflammatory cells, regulates angiogenesis, stimulates transcriptional activation of extracellular matrix synthesis-related genes, plays an important role in the process of wound repair and has been implicated in the pathogenesis of several diseases characterized by abnormal fibrogenesis (1-4).

In mammalian species, there are three known members of the TGF-β family, TGF- [symbol] ₁, TGF- [symbol] ₂ and TGF- [symbol] ₃ (1,2). These isoforms exert similar biological activities in some cell systems, but different activities in other systems (5-7). In the mink lung epithelial cell model system, all three isoforms bind to cell surface TGF- [symbol] receptors with similar affinity and show similar growth inhibitory activity (5-7). They are not equivalent in inhibiting growth of endothelial cells (5-7). In a wound-healing model, TGF- [symbol] ₃ reduces scarring whereas TGF- [symbol] ₁ enhances it (8). The mechanisms by which these isoforms exert different biological activities are not well understood. However, several TGF- [symbol] binding molecules have been reported to be involved in determining the activities of TGF-β isoforms (9-13). Heparin and the highly sulfated liver heparan sulfate potentiate the biological activity of TGF- [symbol] ₁, but not the other isoforms (9). The expression of the TGF [symbol] β type III receptor and an alternatively spliced TGF [symbol] β type II receptor is known to be required for responsiveness to TGF- [symbol] ₂ in several cell types (10). _{.} α₂-Macrogl 2M) can be altered by proteases or primary amines to form so-called activated α₂-Macroglobulin (α₂M*), which interacts differentially with these TGF-β isoforms and contributes to their differential activities in some experimental systems (11-14). Among these TGF-β binding molecules, α₂M* is unique in its ability to bind TGF- [symbol] isoforms with distinct affinities and to affect their plasma clearance (15). [symbol] ₂M* also forms complexes with other growth factors, cytokines and hormones and modulates their biological activities in many experimental systems (16-18).

An active site in TGF [symbol] ₁ and TGF [symbol] ₂ responsible for high-affinity binding to α₂M* has been recently identified at Trp-52 (19). Synthetic peptides containing Trp-52 are capable of blocking the formation of complexes between [symbol] ₂M* and TGF- [symbol] isoforms. They also block the formation of complexes between α₂M* and other growth factors, cytokines and hormones (19).

The inventor has discovered that specific fatty acids (a) strongly inhibit complex formation between [symbol] ₂M and TGF- [symbol] isoforms and (b) induce the dissociation of [symbol] ₂M*-TGF- [symbol] complexes, thereby effectively modulating the activity of TGF-β by providing more free TGF-β. It is further disclosed that fatty acids modulate TGF- [symbol] activity in cells and affect the clearance of TGF- [symbol] ₁-α₂M* and TGF- [symbol] ₂- [symbol] ₂M* complexes from serum.

U.S. Pat. No. 5,147,854 (Newman, Sep. 15, 1992) describes a combination of TGF-β₁, a polyunsaturated fatty acid (PUFA) and a retinoid, which in combination are capable of killing specific human carcinoma and melanoma cell lines. The selected polyunsaturated fatty acids contain two or more double bounds in the hydrocarbon chain. Unsaturated fatty acids and TGF-β alone are taught to be ineffective. It is important to note that Newman uses cells grown in serum-free medium, which does not contain α₂-macroglobulin. Thus, the in vivo efficacy of the TGF-β-PUFA-retinoid combination taught by Newman is not known.

According to the invention disclosed herein, specific fatty acids can be used to potentiate the activities of many growth factors and cytokines such as platelet-derived growth factor AA and BB, vascular endothelial cell growth factor, fibroblast growth factors, interleukins, growth hormone, insulin, insulin-like growth factor 1 and 2, nerve growth factor, neurotrophins and others. All of these growth factors and cytokines are known to be regulated by alpha-2-macroglobulin. According to our invention, specific fatty acids can be used along or in combination of the growth factors or cytokines to treat various diseases in which both growth factors/cytokines and alpha-2-macroglobulin are involved.

U.S. Pat. No. 5,981,606 (Martin, 1999) discloses a combination of pyruvate, lactate, an antioxidant, a mixture of saturated and unsaturated fatty acids, and TGF-β for reducing scaring and increasing proliferation and resuscitation of mammalian cells. The TGF-beta-wound healing compositions taught in the '606 patent to be useful for treating disease via topical application and ingestion. However, no data directly related to wound healing is presented in that specification.

According to the present invention, specific fatty acids exert their biological effects via affecting the interaction of endogenous TGF-β and α-2-macroglobulin, both of which play important roles in the development of many diseases (as described above). In contrast, a mixture of unsaturated and saturated fatty acids described in the '606 patent is used for the repair of cellular membranes and resuscitation of mammalian cells. The pharmacological mechanisms of fatty acids in their and our inventions are in fact completely different. Their invention does not specify fatty acids for better efficacy.

### BRIEF SUMMARY OF THE INVENTION

The inventor has discovered that fatty acids and their derivatives can bind to activated α₂-macroglobulin. The fatty acids, by binding to activated α₂-macroglobulin, prevent activated α₂-macroblobulin from binding to a cognate growth factor. Alternatively, the fatty acids, by binding to a preexisting α₂-macroglobulin-growth factor complex, facilitate the release of the growth factor from the complex. In both scenarios, the addition of a fatty acid to a sample containing an α₂-macroglobulin and a growth factor results in an increase in the amount of free growth factor and thus, effectively an increase in growth factor activity in a sample. An object of this invention is to modulate growth factor activity, especially TGF-β activity, in an animal by administering an effective amount of a fatty acid or a derivative thereof to the animal.

In one embodiment, the invention is drawn to a method for modulating the activity of a growth factor in a sample, which contains an activated α₂-macroglobulin, comprising (a) contacting the sample with a fatty acid in an amount sufficient to inhibit the formation of a complex between the growth factor and the activated α₂-macroglobulin, wherein (b) the fatty acid binds to the activated α₂-macroglobulin. In another embodiment, the invention is drawn to a method for modulating the activity of a growth factor in a sample, which contains an activated α₂-macroglobulin-growth factor complex, comprising (a) contacting the sample with a fatty acid in an amount sufficient to promote the dissociation of the activated α₂-macroglobulin-growth factor complex, wherein (b) the fatty acid binds to the α₂-macroglobulin portion of the activated α₂-macroglobulin-growth factor complex and (c) the growth factor dissociates from activated α₂-macroglobulin. Preferably, the fatty acid, which may be saturated or unsaturated, has a carbon skeleton of at least 14 carbons. The fatty acid may be myristic acid, palmitic acid, stearic acid, arachidonic acid, oleic acid, γ-linolenic acid, linoleic acid, palmitoleic acid or linolenic acid. Representative fatty acids are arachidonic acid or myristic acid.

Given that the inventive step involves the discovery that fatty acid binding to α₂-macroglobulin destabilizes complex formation between activated α2-macroblobulin and a growth factor, the growth factors to which the invention is directed are those growth factors that can bind to activated α₂-macroglobin. Preferred growth factors include platelet-derived growth factor-AA, platelet-derived growth factor-BB, vascular endothelial cell growth factor, fibroblast growth factors, interleukins, growth hormone, insulin, insulin-like growth factor-1, insulin-like growth factor-2, nerve growth factor, neurotrophins and TGF-β, which includes TGF-β₁, TGF-β₂ and TGF-β₃. More preferred growth factors are TGF-βs, preferably TGF-β₁.

The sample to which the fatty acid is added may be in vitro, in situ or in vivo. Preferably the sample is a tissue or blood plasma. The sample may be a tissue or plasma of an animal, including mammals such as mice and humans. More preferably, the sample is a tissue or plasma in an animal.

In another embodiment, growth factor activity in the sample is increased due to growth factor release from activated α₂-macroglobulin upon the addition to a fatty acid to the sample. Alternatively but not exclusively, growth factor activity in the sample is effectively increased due to the inhibition of growth factor binding to activated α₂-macroglobulin upon the addition of a fatty acid to the sample. Preferably, upon addition of a fatty acid to a sample, (a) formation of a complex between the growth factor and activated α₂-macroglobulin in a sample is inhibited at least 10% or (b) dissociation of a complex between the growth factor and α₂-macroglobulin in a sample is increased at least 10%, relative to an equivalent sample which did not receive the fatty acid.

### BRIEF DESCRIPTION OF THE DRAWINGS

FIG. 1 Inhibition of 125I-TGFβ [symbol] 1 and [symbol] 2M* complex formation by saturated (A) and unsaturated (B) fatty acids. α2M* was preincubated with various concentrations as indicated of saturated fatty acids (n-caprylic acid, lauric acid, myristic acid, palmitic acid and stearic acid) and unsaturated fatty acids (oleic acid, palmitoleic acid, linolenic acid,_{.} γ-linolenic acid, linoleic acid and arachidonic acid) for 30 min at room temperature and reacted with ¹²⁵I-TGF [symbol] β₁. After 30 min at room temperature, the reaction mixtures were analyzed by 5% non-denaturing PAGE and autoradiography (a). The arrow indicates the location of the ¹²⁵I-TGF [symbol] β_{1.}-α₂M* complex which was quantified by a PhosphoImager (b). Data are representative of four similar experiments.

FIG. 2 Effects of arachidonic acid derivatives and analogues on ¹²⁵I-TGF [symbol] β₁- [symbol] ₂M* complex formation. α₂M* was preincubated with various concentrations as indicated of arachidonic acid (AA) arachidonic acid methyl ester (AA-O-Me) and analogue (ETYA; 8, 11, 14 eicosatien-5-ynoic acid) for 30 min at room temperature. ¹²⁵I-TGF- [symbol] ₁ was then added to the reaction mixture. After 30 min at room temperature, the reaction mixtures were analyzed by 5% non-denaturing PAGE and autoradiography (a). The arrow indicates the location of the ¹²⁵I-TGF [symbol] β₁- [symbol] ₂M* complex which was quantified by a PhosphoImager (b). Data are representative of four similar experiments.

FIG. 3 Effects of myristic acid and arachidonic acid on formation of ¹²⁵I-TGF [symbol] β isoform and α₂M* complexes identified on non-denaturing PAGE (A) and SDS-PAGE (B). α₂M* was preincubated with various concentrations as indicated of myristic acid and arachidonic acid for 30 min at room temperature and reacted with ¹²⁵I-TGF- [symbol] ₁, ¹²⁵I-TGF- [symbol] ₂ or ¹²⁵I-TGF min at room temperature, the reaction mixtures were analyzed by 5% non-denaturing PAGE (A) or 7.5% SDS-PAGE following cross linking by DSS (B) and autoradiography (a). The arrow indicates the location of the ¹²⁵I-TGF- [symbol] - [symbol] ₂M* complex which was quantified by a PhosphoImager (b). Data are representative of four similar experiments.

FIG. 4 Dissociation of ¹²⁵I-TGF [symbol] β₁- and [symbol] ₂M* and ¹²⁵I-TGF [symbol] ₂-₂M* comp arachidonic acid. α₂M* was reacted separately with ¹²⁵I-TGF- [symbol] ₁ and ¹²⁵I-TGF- [symbol] ₂ for room temperature. The reaction mixture was then treated with various concentrations as indicated of arachidonic acid. After 30 min at room temperature, the reaction mixtures were analyzed by 5% non-denaturing PAGE and autoradiography (a). The arrow indicates the location of the ¹²⁵I-TGF- [symbol] ₁ and α₂M* or ¹²⁵I-TGF- [symbol] ₂- [symbol] ₂M* PhosphoImager (b). Data are representative of four similar experiments.

FIG. 5 Gel filtration chromatography of ³H-arachidonic acid-₂M* complexes. ³H-Arachidonic acid (³H-AA) was preincubated with and without [symbol] ₂M* (which had been activated by methylamine), or with native [symbol] ₂M. After 30 min at room temperature, the reaction mixture was applied onto a column (0.7×40 cm) of Sephacryl S-300 HR. The fractional volume was 1 ml. The ³H-radioactivity in the fractions was determined by scintillation counting. _{.} α₂M* and native α₂M in the fractions were identified by Coomassie blue staining (Inset). The arrow indicates the location of α₂M*. Data are representative of three similar experiments.

FIG. 6 Arachidonic acid reversal of the [symbol] ₂M* inhibitory effect on ¹²⁵I-TGF-β₂ binding to TGF- [symbol] receptors (A) and TGF- [symbol] ₂-induced growth inhibition (B) and transcriptional activation (C) in Mv1Lu cells. (A) [symbol] ₂M* (200 μg/ml) was preincubated with arachidonic acid (AA) (0 or 30 μM) and various concentrations (0, 1.25, 2.5, 5 and 10 pM) of ¹²⁵I-TGF-β₂ with and without TGF [symbol] β peptantagonist (30 μM) (19). After 30 min at room temperature, the ¹²⁵I-TGF-β₂ solutio was added to the medium and the ¹²⁵I-TGF-β₂ binding was determined after 2.5 hr at 0° C. The binding of ¹²⁵I-TGF-β₂ obtained in the presence of [symbol] ₂M* was mainly non-specific binding of ¹²⁵I-TGF- [symbol] since it was not further inhibited by the presence of TGF [symbol] β peptantagonist. Data are representative of four similar experiments. (B) Cells were treated with various concentrations of TGF- [symbol] ₂ in the presence and absence of [symbol] ₂M* (200 μg/ml) and arachidonic acid (AA) (0.5 or 1.0 μM). After 18 hr at 37° C., the [methyl-³H]-thymidine incorporation into cellular DNA of cells was determined. The [methyl-³H]-thymidine incorporation in cells treated without TGF [symbol] β₂ and arachidonic acid was taken as 0% inhibition. Data are representative of four similar experiments. (C) Cells transiently transfected with the p3TP plasmid were treated with various concentrations of TGF- [symbol] ₂ in the presence and absence of α₂M* (200 μg/ml) and arachidonic acid (AA) (12.5 and 25 μM). After 12 hr at 37° C., the luciferase activity of the cell extracts was determined and expressed as arbitrary units (A.U.). Data were obtained from three different experiments; values are mean SD (*, P<0.05 vs luciferase activity of cells treated with α₂M* and TGF [symbol] β₂).

FIG. 7 Plasma clearance of ¹²⁵I-TGF [symbol] β₁ (A) or ¹²⁵I-TGF [symbol] β₂ (B) treated with [symbol] _{presence and absence of arachidonic acid.} ¹²⁵I-TGF [symbol] β₁ (A) or ¹²⁵I-TGF- [symbol] ₂ (B) was incubated with α₂M* in the presence and absence of arachidonic acid (AA). After 30 min at room temperature, the ¹²⁵I-TGF [symbol] β₁ or ¹²⁵I-TGF [symbol] β₂ solution was injected into the tail veins of mice Blood samples were collected at the time intervals indicated. The radioactivity in the blood sample collected 10 seconds after i.v. injection of the isotope solution was taken as 100%. Data are representative of four similar experiments.

### DETAILED DESCRIPTION OF THE INVENTION

The activity and plasma clearance of many growth factors and cytokines, including TGF-β, are known to be regulated by activated α₂-macroglobulin (α2M*). The inventor has discovered that fatty acids are capable of inhibiting complex formation of [symbol] 2M* and representative growth factors/cytokines, e.g., platelet-derived growth factor-AA, platelet-derived growth factor-BB, vascular endothelial cell growth factor, fibroblast growth factors, interleukins, growth hormone, insulin, insulin-like growth factor-1, insulin-like growth factor-2, nerve growth factor, neurotrophins and TGF- [symbol] isoforms, as demonstrated by non-denaturing and SDS-polyacrylamide gel electrophoresis. The inventor has also discovered that fatty acids are capable of disrupting preexisting [symbol] ₂M*-growth factor/cytokine complexes. This complex-inhibition or complex-disruption activity of fatty acids is dependent on carbon chain length (C20, C18, C16, C14>C12>C10), degree of unsaturation (polyunsaturated>saturated) and growth factor (e.g., TGF- [symbol] ₁>TGF [symbol] β₂>TGF-β₃). Arachidonic acid, which is one of the most potent inhibitors, is also capable of dissociating TGF- [symbol] - [symbol] 2M* complexes but higher concentrations are required. Arachidonic acid appears to inhibit TGF- [symbol] β-α2M* complex formation by binding specifically to _{.} α₂M* as demonstrated by gel filtration chromatography. Arachidonic acid reverses the inhibitory effect of _{.} α₂M* on TGF [symbol] β binding, TGF- [symbol] -induced growth inhibition and transcriptional activation in mink lung epithelial cells and affects plasma clearance of TGF [symbol] --2M* complexes in mice. These results show that fatty acids are effective modulators of growth factor/cytokine activity and plasma clearance.

TGF [symbol] β is a potent growth factor, which has been the subject of intense study because of its role in diverse biological processes and its potential role in disease states. It exerts various biological activities with optimal concentrations in the picomolar range. Some of its activities are regulated at the transcriptional level and others are regulated post-transcriptionally. Post-translational control is also prominent and includes activation of latent TGF- [symbol] and modulation by TGF [symbol] β binding molecules such as [symbol] ₂M*, betaglycan, decorin, thrombospondin, fetuin, and latent TGF- [symbol] binding protein (11,12,31-36). The mechanisms of in vivo activation of latent TGF- [symbol] are not well understood, but it is generally believed that latent TGF [symbol] β is activated both by proteolysis at the cell surface and by acidic pH in endosomal compartments (34,35). The TGF- [symbol] binding molecules modulate TGF [symbol] β activities by inhibiting its binding to TGF [symbol] β receptors and/or by sequestering TGF [symbol] β molecules in the extracellular space. One such binding agent is α₂M*, which affects TGF- [symbol] activities by forming a complex that does not bind to TGF- [symbol] receptors in cells. _{.} α₂M* neutralizes TGF [symbol] β activities in many experimental systems (13,16-18) but, unlike other TGF- [symbol] modulators, [symbol] ₂M* is also involved in plasma clearance of TGF- [symbol] (15)._{.} αmajor plasma binding protein for TGF- [symbol] and the [symbol] ₂M* receptor mediates plasma clearance of the TGF [symbol] β-α₂M* complex (12,15,30).

The exact molecular mechanisms by which [symbol] ₂M* forms complexes with TGF- [symbol] and many other factors that do not share amino acid sequence homology with TGF- [symbol] are presently not well defined in the art. The inventor hypothesizes that [symbol] ₂M* forms complexes with TGF [symbol] α and these factors via non-covalent hydrophobic interactions with topologically diverse exposed molecular surfaces which do not have consistent amino acid motifs. Several facts, which the inventor has applied to the conceptual formulation of the inventive step, include (a) TGF- [symbol] peptides containing the residue Trp-52 are potent inhibitors of complex formation between α₂M* and TGF [symbol] β and other growth factors (19); (b) replacement of Trp-52 with alanine completely abolishes the inhibitory activity of the TGF [symbol] β peptides however, replacement of the residue Trp-52 with hydrophobic amino acids such as phenylalanine and leucine leaves its inhibitory activity largely intact, 19); and (c) a hydrophobic small peptide whose amino acid sequence is derived from [symbol] ₂M* blocks complex formation of α₂M* and both TGF- [symbol] and PDGF (37). According to the present invention, fatty acids are potent inhibitors of TGF [symbol] β- [symbol] ₂M* complex formation. It is further disclosed herein that arachidonic acid binds to [symbol] ₂M* but not native [symbol] ₂M, in further support of this hypothesis. However, it is herein disclosed that the inhibitory effect of fatty acids requires the presence of a free carboxyl group in addition to hydrophobicity at the binding site. It appears that [symbol] ₂M* contains high-affinity hydrophobic regions (pockets or cavities) that can specifically interact with hydrophobic subdomains of TGF- [symbol] and other factors. The hydrophobic subdomains of TGF- [symbol] located on the molecule surface possibly include Trp-52 and other neighboring hydrophobic amino acid residues. The evidence disclosed here in the working examples indicates that fatty acids with [symbol] ≧14 carbon atoms and double bonds (e.g., arachidonic acid) bind to the proposed putative pocket or cavity in the α₂.M* molecule with high affinity.

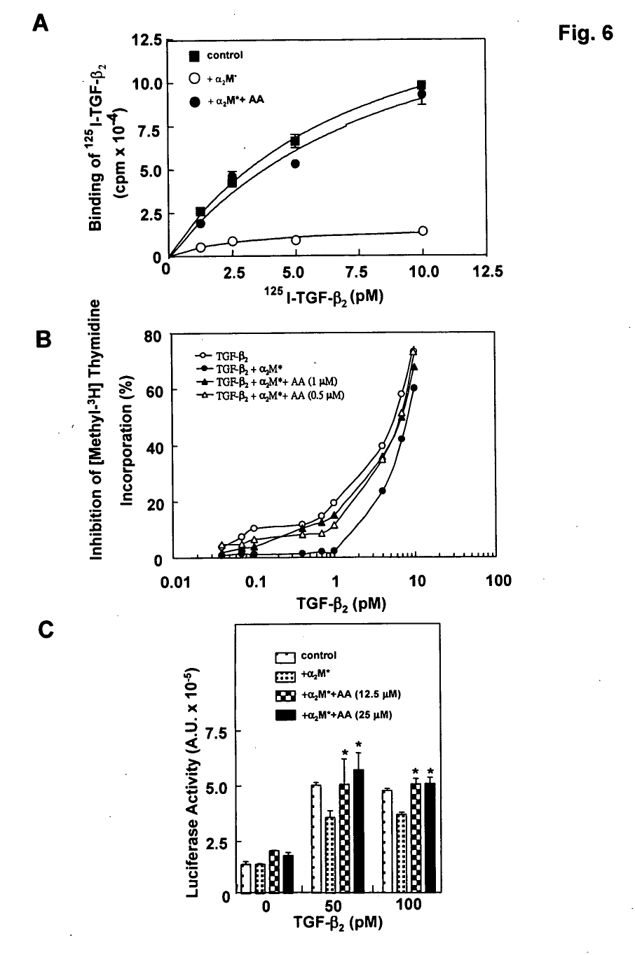

Since low levels of active TGF- [symbol] in plasma have been implicated in the pathogenesis of atherosclerosis and since it also is involved in wound repair and tissue fibrosis (1-4), the identification of substances, such as the fatty acids of the instant invention, that can alter these biological effects may be important therapeutically. In preliminary studies conducted by the inventor, oral administration of fatty acids to humans suffering psoriasis has resulted in amelioration of symptoms. Compounds that are capable of blocking and/or dissociating TGF- [symbol] -α₂M* complexes, thereby affecting the levels of free TGF- [symbol] in plasma and tissues, have therapeutic potential as systemic or regionally-delivered drugs for many common diseases. It is herein disclosed that endogenous fatty acids are potent inhibitors of complex formation of TGF- [symbol] and α₂M*. The IC₅₀s (7.8±1.4 and 9.1_{.}±0.5 μM) of arachidonic acid and myristic acid well below their critical micelle concentrations (20 μM and >1 mM, respectively) (27,28). It is also disclosed that arachidonic acid is capable of modulating TGF- [symbol] binding and TGF- [symbol] activity in mink lung epithelial cells in the presence of bovine serum albumin (FIG. 6A) and fetal calf serum (FIGS. 6B and C). This is consistent with the known physiological role of serum albumin in the transport of free fatty acids to high-affinity binding sites on other protein (e.g., [symbol] ₂M*) and supports the physiological relevance of the observation that arachidonic acid modulates TGF [symbol] β activity in environments containing serum albumin. Human serum albumin (HSA) plays an essential role as a transporter of fatty acids. The plasma concentration of HSA is approximately 0.6 mM and the molar ratio of fatty acids and HSA is approximately 0.5 to 2.0, depending on conditions (e.g., fasting) (38). The plasma concentration of free fatty acids may be elevated and reach μM concentrations under certain pathophysiological conditions (injury, fasting, stress, heparin administration, diabetes, bacterial infection and others) (38,39). The IC₅₀s of most of the fatty acid examined for inhibiting TGF- [symbol] binding to [symbol] ₂M* are <10 μM. These concentrations cań occur at sites of injury (wound) or inflammation. Fatty acids are known to be generated locally at considerably higher concentrations than the mean blood levels. In the interstitial space, where albumin concentration is much lower than within the blood, fatty acids may modulate TGF- [symbol] activity even more significantly than in plasma. Fatty acids (e.g., arachidonic acid) have also been found to block complex formation between α₂M* and nerve growth factor (NGF) and basic fibroblast growth factor (bFGF) in the laboratory. This suggests that exogenous fatty acids (e.g., polyunsaturated fatty acids including those not found in natural products) can be designed to potentiate TGF [symbol] β and other growth factor/cytokine/hormone activities in order to treat human or animal diseases (16-18).

As discussed above, it is well known in the art that both α-2-macroglobulin and TGF-β are involved in many pathophysiological processes, such as injury, inflammation, arteriosclerosis, autoimmune diseases, psoriasis, Alzheimer disease and others. According to the present invention, specific polyunsaturated fatty acids, for example linolenic acids, which are known to exhibit no toxicity to humans or animals, can be used to treat these and other diseases via topical application or ingestion. Fatty acids may be used alone or in combination with other ingredients for topical application, such as to a wound, or for oral ingestion for treating various diseases ranging from psoriasis to Alzheimer disease. It is known in the art that endogenous TGF-β is good for alleviating these diseases. Specific fatty acids can modulate, i.e. increase or decrease, the endogenous TGF-β activity through their effect on the interaction of TGF-β and α₂-macroglobulin.

The fatty acids of the instant invention may be added to a sample in an amount to sufficient to facilitate a change in the amount of free growth factor, i.e. not bound to α2-macroglobulin, in a sample. The change in free growth factor is proportional to the concentration of free growth factor in a sample after the addition of fatty acid minus the concentration of free growth factor in the same or similar sample before the addition of fatty acid. Alternatively or additionally, the fatty acids of the instant invention may be added to a sample in an amount to sufficient to facilitate a change in the concentration of growth factor-α2-macroglobulin complexes in the sample. The change in concentration of complexes is proportional to the concentration of complexes in a sample after the addition of fatty acid minus the concentration of complexes in the same or similar sample before the addition of fatty acid. The percent change in complex formation is calculated as ([pre-fatty acid complex]—[post-fatty acid complex])/[pre-fatty acid complex].

As used herein, the term “modulation” or “modulating the activity of a growth factor” means effecting a change in the activity of a growth factor in a sample relative to a baseline of activity. The change in activity may be an increase in growth factor activity or a decrease in growth activity relative to the baseline. The baseline of growth factor activity is the growth factor activity in a sample similar to the sample that receives the fatty acid, but which does not receive the fatty acid. Alternatively, the baseline of growth factor activity is the growth factor activity in the sample just prior to the administration of the fatty acid.

As used herein, the term “sample” means any mixture, solution, ex vivo tissue, in vivo tissue, blood, plasma, serum, biological extract, cellular extract, intact cell, interstitial space, mucosa, skin, skin surface or extracellular matrix. The preferred sample contains an α₂-macroglobulin or is in close proximity to an area, tissue or other sample that contains an α₂-macroglobulin. A preferred sample is from or in an animal. A preferred animal is a human.

As used herein, the phrase “inhibit the formation of a complex” refers to the prevention of the binding of a growth factor to an α₂-macroglobulin molecule as a result of the binding of a fatty acid to the α2-macroglobulin. As used herein, the phrase “inhibited at least 10% (or 20%, 40% or 60%, as the case may be)” refers to a 10% (or 20%, 40% or 60%, as the case may be) change in the concentration of growth factor/α₂-macroglobulin complex upon the addition of a fatty acid. For example, percent inhibition may be determined according to eq. 1, wherein [complex₀] is the concentration of a growth factor/α₂-macroglobulin complex in a sample in the absence of the fatty acid, and [complex₁] is the concentration of a growth factor/α₂-macroglobulin complex in a sample in the presence of the fatty acid:

$$eq . 1 \text{:} \qquad percent inhibition = \frac{[ {complex}_{0} ] - [ {complex}_{1} ]}{[ {complex}_{0} ]}$$

As used herein, the term “growth factor” means any hormone, growth factor, cytokine, extracellular matrix component or any cell-signaling molecule that binds to activated α₂-macroglobulin. A preferred embodiment of growth factor is TGF-β.

As used herein, the term “fatty acid” means a molecule having a hydrocarbon chain and a terminal carboxyl group. The hydrocarbon chain may be saturated, i.e., having only single bonds between carbons, or unsaturated, i.e., having one or more double or triple bonds between carbons. As used herein, fatty acids may comprise further substituents or pendant groups or may be salts or derivatives of fatty acids. Fatty acids include myristic acid, palmitic acid, stearic acid, arachidonic acid, oleic acid, γ-linolenic acid, linoleic acid, palmitoleic acid and linolenic acid. Preferred fatty acids include myristic acid and arachidonic acid, or their derivatives.

The following working examples are provided to illustrate and support the claims of the invention and are not intended to limit the scope of the claims.

### EXAMPLE 1

### Fatty Acids Block Complex Formation of TGF- [symbol] ₁ and [symbol] ₂M

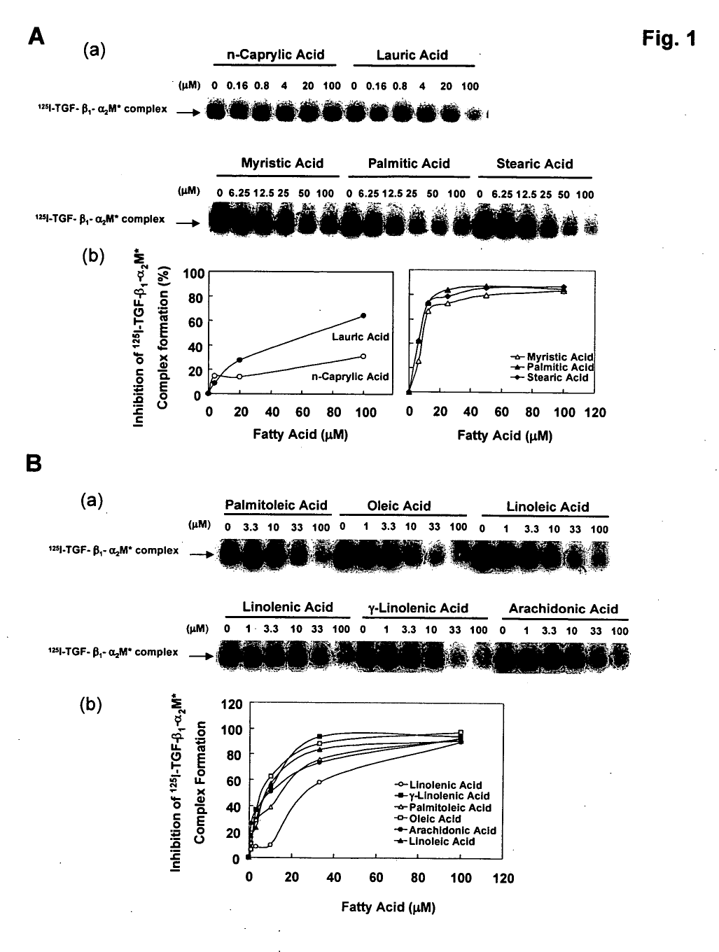

Saturated and unsaturated fatty acids are present in plasma and tissues (25,26). The effects of various concentrations of saturated fatty acids on the formation of complexes between ¹²⁵I-TGF [symbol] β₁ and [symbol] ₂M* were examined. ¹²⁵I-TGF- [symbol] ₁ (1 nM) was incubated μg/ml) in the presence of various concentrations of n-caprylic acid (10 carbon atoms), lauric acid (12 carbon atoms), myristic acid (14 carbon atoms), palmitic acid (16 carbon atoms) and stearic acid (18 carbon atoms). After 30 min at room temperature, the reaction mixture was analyzed by 5% non-denaturing PAGE and autoradiography, a standard method form determining complex formation between TGF-β and α₂M* (12). In this system, the complexes of [symbol] ₂M* and various ¹²⁵I-labeled interacting proteins co-migrate with [symbol] ₂M* (which migrates slowly in the separating gel due to the large size of the molecule) whereas the free ¹²⁵I-labeled proteins migrate at the dye front or do not migrate into the separating gel depending upon its acidity or basicity at the electrophoresis buffer pH 8.3. For example, ¹²⁵I-TGF [symbol] β does not migrate into the separating gel due to its basicity under the electrophoretic conditions (pH 8.3). As shown in FIG. 1A, these saturated fatty acids inhibited the formation of complexes between TGF- [symbol] ₁ and α₂M* in a concentration-dependent manner with IC₅₀s of 6.6±0.9 (n=4), 8.5±1.0 (n=4) and 9.1 (n=4), and 68_{.}±10 (n=4) μM for stearic acid, palmitic acid, myristic acid and lauric acid, respectively. n-Caprylic acid was a relatively weak inhibitor. At 100 μM, it inhibited 20% of the complex formation between TGF [symbol] β₁ and α₂M*. Esterification consistently abolished the inhibitory activities of the fatty acids. These results suggest that many saturated fatty acids are capable of inhibiting the complex formation bewteen ¹²⁵I-TGF [symbol] ₁ and α₂M* but require a minimum carbon chain length approximately 14 and the presence of a free carboxyl group for optimal activities.

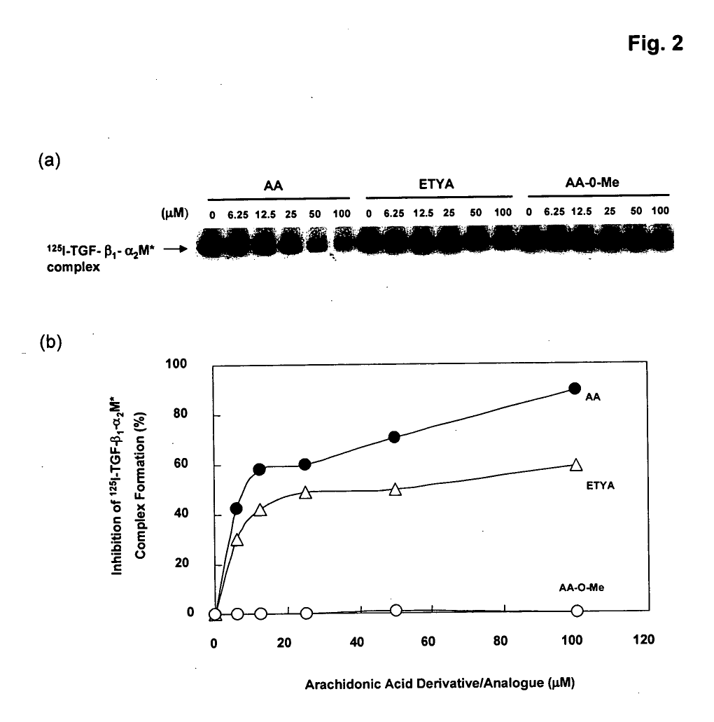

As shown in FIG. 1A, myristic acid, palmitic acid and stearic acid, which contain 14, 16 and 18 carbon atoms, respectively, potently inhibited complex formation of ¹²⁵I-TGF- [symbol] ₁ and [symbol] ₂M*. Various unsaturated fatty acids, which have the same carbon chain length because double bonds are known to shorten the molecular length of fatty acids and confer more rigid configurations, were tested. As shown in FIG. 1B, arachidonic acid (20:4n6), oleic acid (18:1n9), γ-linolenic acid (18:3n6), linoleic acid (18:2n6), palmitoleic acid (16:1n7), and linolenic acid (18:3n3) inhibited complex formation of ¹²⁵I-TGF- [symbol] ₁ and [symbol] ₂M* in a concentration-dependen manner with IC₅₀s of 7.8±1.4 (n=3), 5.2±2.0 (n=3), 8.0±2.0 (n=3), (n=3) and 26±3.1 (n=3) μM, respectively. The activities of most of these unsaturated fatty acids were similar to those of their saturated counterparts of identical chain length (arachidonic acid, linoleic acid and_{.}-linolenic acid), but, linolenic and palmitoleic acids were weaker than their saturated counterparts. It is important to note that ω-6 fatty acids (arachidonic acid, γ-linolenic acid and linoleic acid) were more potent than ω-3 fatty acids (e.g., linolenic acid). Since arachidonic acid was one of the most potent inhibitors among the fatty acids tested, we studied the structure and function relationship of arachidonic acid by examining the effects of arachidonic acid derivatives and analogs including a nonmetabolic analog ETYA (8, 11, 14 eicosatrien-5-ynoic acid), arachidonic acid methyl ester, and its 20-, 15-, and 5-hydroxy derivatives on the formation of complexes between ¹²⁵I-TGF- [symbol] ₁ and [symbol] ₂M*. As shown in FIG. 2, ETYA (IC₅₀: 30_{.}±3.0 μM) was less effective than arachidonic acid in inhibiting complex formation of ¹²⁵I-TGF [symbol] and [symbol] ₂M*, whereas arachidonic acid methyl ester was inactive. The hydroxy derivatives of arachidonic acid showed very weak activities (data not shown). The IC₅₀s of these derivatives were estimated to be >100 μM. These results indicate that replacement of the double bond with the triple bond, esterification of the carboxy group and addition of a hydroxy group in the hydrocarbon chain all significantly diminish the ability of arachidonic acid to inhibit complex formation between TGF [symbol] β₁ and α₂M*.

### EXAMPLE 2

### Fatty Acids Inhibit Complex Formation of TGF- [symbol] Isoforms and [symbol] ₂M*

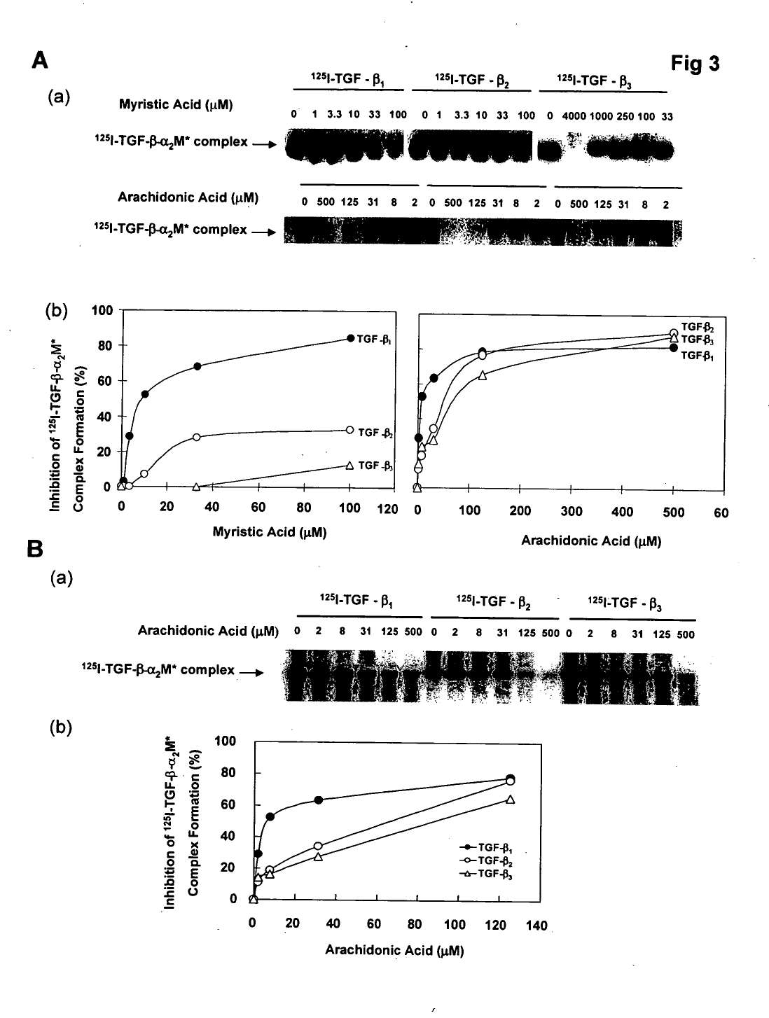

TGF- [symbol] isoforms bind to α₂M* with different affinities: TGF [symbol] β₂>TGF [symbol] β₁ ( active sites of TGF [symbol] β₁ and TGF- [symbol] ₂ responsible for high-affinity binding to [symbol] ₂M* are disti from the low-affinity [symbol] ₂M* binding site in TGF- [symbol] ₃ (19). To determine if fatty acids differentially affect the binding of TGF- [symbol] isoforms to [symbol] ₂M*, the effects of various concentrations of arachidonic acid and myristic acid on complex formation of ¹²⁵I-labeled TGF [symbol] β isoforms and [symbol] ₂M were determined*. Myristic acid and arachidonic acid were the most potent inhibitors of complex formation among the saturated and unsaturated fatty acids that were tested. As shown in FIG. 3A, myristic acid inhibited complex formation of_{.} α₂M* and ¹²⁵I-TGF- [symbol] ₂ or TGF- [symbol] ₃ much less than that of [symbol] ₂M* and TGF [symbol] β₁. It inhibited 30% of the complex form _{.} α₂M* and TGF [symbol] β₂ and TGF [symbol] β₃ at 100 and >250 μM, respectively. Arachidon polyunsaturated fatty acid, was a stronger inhibitor of complex formation of [symbol] ₂M* and TGF-_{.}β₂/TGF [symbol] β₃. It inhibited 50% of the complex formation of [symbol] ₂M* and ¹²⁵I-TGF- [symbol] ₂ at 50 μM (FIG. 3A). The observation that myristic acid and arachidonic acid inhibited complex formation of ¹²⁵I-TGF- [symbol] ₂ and_{.} α₂M* more weakly than they inhibited complex formation of ¹²⁵I-TGF [symbol] β₁ and α₂M* is consistent with the binding affinity data. TGF- [symbol] [symbol] ₂M* with higher affinity than TGF [symbol] β₁ (14). To further define the inhibitory effect of fatty acids on complex formation of TGF [symbol] β isoforms and [symbol] ₂M*, the ¹²⁵I-TGF [symbol] β isoform- [symbol] ₂were cross-linked by a cross-linking agent (DSS) following incubation of ¹²⁵I-TGF [symbol] β isoforms and [symbol] ₂M* in the presence of various concentrations of arachidonic acid. The cross-linked ¹²⁵I-TGF [symbol] β isoform- [symbol] ₂M* complexes in the reaction mixtures were then analyzed by 7.5% SDS-PAGE and autoradiography. As shown in FIG. 3B, arachidonic acid blocked complex formation of ¹²⁵I-TGF [symbol] β isoforms and [symbol] ₂M* with effective concentrations comparable to those obtained by determining ¹²⁵I-TGF- [symbol] isoform- [symbol] ₂M* complex formation with non-denaturing PAGE (FIG. 3A).

### EXAMPLE 3

### Fatty Acids are Capable of Dissociating TGF- [symbol] [symbol] -₂M Complexes

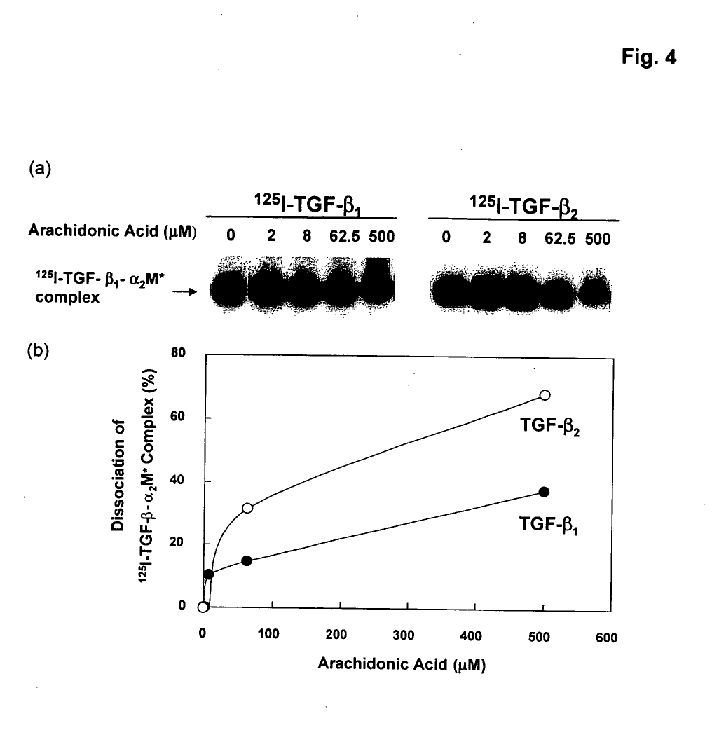

To determine whether fatty acids are capable of dissociating TGF- [symbol] - [symbol] ₂M* complexes, various concentrations of arachidonic acid were added to a reaction mixture containing ¹²⁵I-TGF- [symbol] ₁ or ¹²⁵I-TGF [symbol] β₃ and α₂M* which had been preincubated at room temperature for 30 mi 30 minutes at room temperature, the ¹²⁵I-TGF [symbol] β isoform-α₂M* complexes in the reaction mixtures were analyzed by 5% non-denaturing PAGE. As shown in FIG. 4, arachidonic acid was able to dissociate the ¹²⁵I-TGF [symbol] β₁- [symbol] ₂M* and ¹²⁵I-TGF- [symbol] ₂- [symbol] ₂and 250 μM, respectively. It is of interest to note that arachidonic acid was more effective in dissociating the ¹²⁵I-TGF- [symbol] ₂- [symbol] ₂M* complex than the ¹²⁵I-TGF-β₁- [symbol] ₂contrast to the observation that arachidonic acid inhibited complex formation ¹²⁵I-TGF- [symbol] ₁ and [symbol] ₂M* more effectively than ¹²⁵I-TGF [symbol] β₂ and [symbol] ₂M*. However, lower concentrations of arachidonic acid were effective in inhibiting complex formation of ¹²⁵I-TGF- [symbol] ₁ and α₂M* than were required to dissociate the ¹²⁵I-TGF- [symbol] ₁- [symbol] ₂M* complex. Myristic acid and other saturated fatty acids were inactive for dissociating the ¹²⁵I-TGF- [symbol] - [symbol] ₂M* complexes at 250 μM.

### EXAMPLE 4

### Arachidonic Acid Binds to α₂M* but not Native [symbol] ₂M

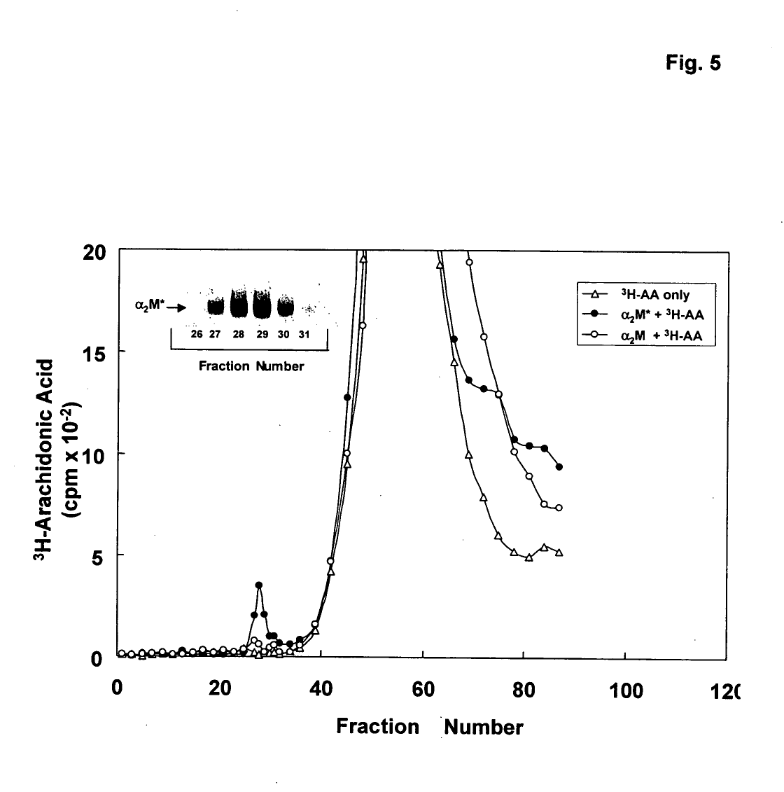

The interaction of ³H-arachidonic acid and α₂M* was determined using gel filtration. ³H-arachidonic acid was incubated with native [symbol] ₂M or [symbol] ₂M*, which was activated by methylamine. After incubation at room temperature for 30 min, the reaction mixture was subjected to gel filtration chromatography on Sephacryl® S-300 HR. The ³H-arachidonic acid radioactivity and concentrations of_{.} α₂M* or native [symbol] ₂M in the eluents were determined by scintillation counting and 5% SDS-PAGE followed by Coomassie blue staining, respectively. As shown in FIG. 5, the reaction mixture containing ³H-arachidonic acid and α₂M* yielded one small and one large ³H-radioactivity peaks after being subjected to gel filtration chromatography on Sephacryl® S-300 HR. The small peak, which appeared in the flow-through fractions, contained the ³H-arachidonic acid- [symbol] ₂M* complex and free [symbol] ₂M*, which was identified by Coomassie blue staining (FIG. 5, inset). The subsequent large peak, which appeared in the column bed volume fractions, was identified as free ³H-arachidonic acid. In contrast, the reaction mixture containing native α₂M and ³H-arachidonic acid showed only the large peak, indicating no complex formation. Under the gel filtration conditions, the stoichiometry of the ³H-arachidonic acid and [symbol] ₂M* complex was estimated to be approximately 2:1. _{.} α₂M*, which was activated by plasmin, was also found to form the ³H-arachidonic acid complex with the similar stoichiometry. These results suggest that arachidonic acid is capable of forming complexes with [symbol] ₂M* but not native_{.} α₂M. Arachidonic acid appears to block complex formation of TGF- [symbol] and [symbol] ₂M* by specific binding to [symbol] ₂M*.

### EXAMPLE 5

### Fatty Acids Block the Inhibitory Effect of [symbol] ₂M* on TGF- [symbol] Binding to TGF [symbol] β Receptors, TGF- [symbol] -Induced Growth Inhibition and Transcriptional Activation in Mv1Lu Cells

Fatty acids, such as myristic acid and arachidonic acid, are present in plasma and other tissues and their levels significantly increase during injury, inflammation and fibrosis (25-28). The levels of TGF [symbol] β and [symbol] ₂M* also increase dramatically. α₂M* is capable of inhibiting TGF- [symbol] activity by forming complexes with TGF- [symbol] and thus preventing it from binding to TGF [symbol] β receptors in cells involved. Fatty acids may potentiate TGF [symbol] β activity by blocking complex formation of [symbol] ₂M* and TGF [symbol] β under these conditions. To test this possibility, we determined the effects of arachidonic acid on ¹²⁵I-TGF- [symbol] ₂ binding (in the presence and absence of α₂M*) to Mv1Lu cells. [symbol] ₂M* is known to inhibit TGF- [symbol] ₂ more strongly than TGF [symbol] β₁ binding to T receptors in cells (13). Various concentrations of ¹²⁵I-TGF- [symbol] ₂ were preincubated with 200 μg/ml of [symbol] ₂M* in the presence or absence of 30 μM arachidonic acid for 30 min prior to the performance of binding assays in Mv1Lu cells. As shown in FIG. 6A, [symbol] ₂M* strongly inhibited ¹²⁵I-TGF-β₂ binding to Mv1Lu cells. The residual ¹²⁵I-TGF- [symbol] binding associated with the cells after [symbol] ₂M* inhibition was mainly due to non-specific binding of ¹²⁵I-TGF- [symbol] ₂. In fact, α₂M* 200 μg/ml completely inhibited the specific binding of ¹²⁵I-TGF- [symbol] ₂ to those epithelial cells as previously reported (13). The inhibition by [symbol] ₂M* was completely reversed by 30 μM of arachidonic acid. To clarify the biological relevance of this observation, the effect of arachidonic acid on the inhibitory effect of_{.} α₂M* on growth inhibition and TGF [symbol] β₂-induced transcriptional activation in Mv1Lu cells was examined._{.} α₂M* has been shown to be effective in blocking TGF- [symbol] ₂-induced growth inhibition (13). As shown in FIG. 6B, TGF- [symbol] ₂ inhibited [methyl-³H]-thymidine incorporation into DNA of Mv1Lu cells in a dose-dependent manner. In the presence of 200 μg/ml of [symbol] ₂M*, the dose-response curve of TGF [symbol] β₂ shifted to the right. In the absence of [symbol] ₂M*, TGF- [symbol] ₂ (1 pM) inhibited approximately 25% of [methyl-³H]-thymid incorporation into DNA of these epithelial cells; this was completely abolished by the presence of α₂M* in the medium. Addition of arachidonic acid at 0.5 and 1 μM reversed the inhibitory effect of_{.} α₂M* on TGF- [symbol] ₂-induced growth inhibition as measured by [methyl-³H]-thymidine incorporation. One μM of arachidonic acid almost completely reversed the inhibitory effect of _{.} α₂M* on growth inhibition induced by 1 pM of TGF- [symbol] ₂. In the absence of α₂M*, arach acid did not affect growth inhibition induced by TGF [symbol] β₂ under the experimental conditions.

One of the prominent biological activities of TGF- [symbol] is transcriptional activation of plasminogen activator inhibitor-1 (PAI-1) and fibronectin (1-4). The effect of fatty acids on the inhibition by_{.} α₂M* of a TGF- [symbol] -responsive promoter construct p3TP-Lux was determined in transfected Mv1Lu cells. The p3TP-Lux contains the PAI-1 promoter and 3 repeats of a phorbol-12-myristate-13-acetate (TPA)-responsive element (29). As shown in FIG. 6C,_{.} α₂M* (200 μg/ml) inhibited approximately 40% of the luciferase activity induced by TGF- [symbol] ₂ (50 and 100 pM). This [symbol] ₂M* inhibition of the TGF [symbol] -induced luciferase activity was completely reversed by either 12.5 or 25 μM of arachidonic acid. In the control experiments, arachidonic acid (12.5 and 25 μM) did not influence the luciferase activity in cells treated with and without TGF- [symbol] ₂ in the absence of [symbol] ₂M*. Together with the results described above, this suggests that fatty acids are capable of modulating the biological activities of TGF- [symbol] under conditions where_{.} α₂M* is present.

### EXAMPLE 6

### Fatty Acids Block α₂M*-Mediated Plasma Clearance of TGF [symbol] β₁ and TGF [symbol] β₂

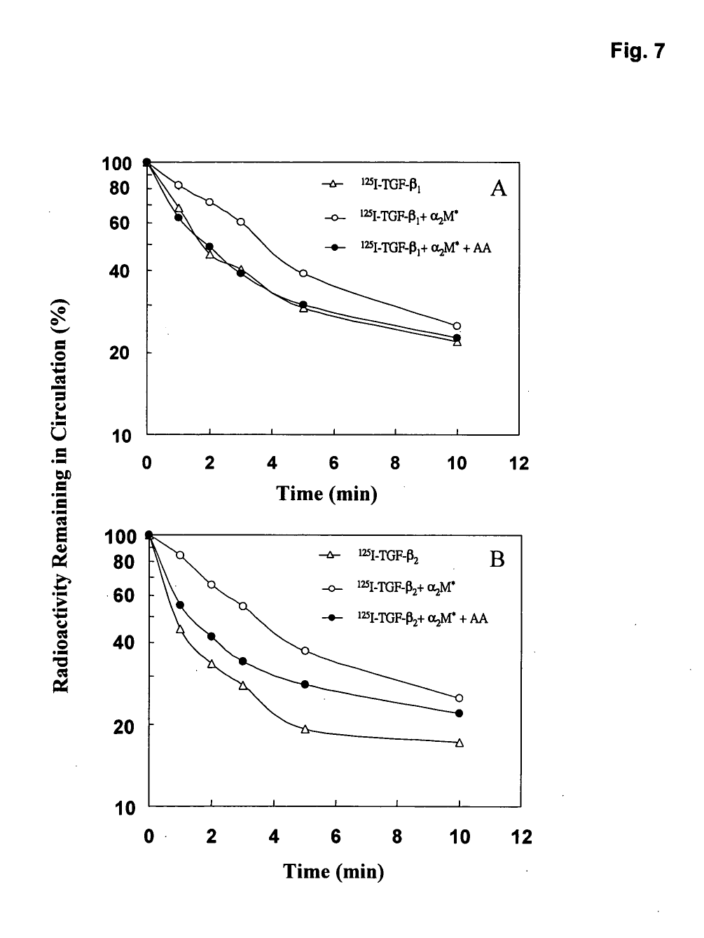

α₂M* has been shown to be involved in plasma clearance of TGF- [symbol] ₁ and TGF- [symbol] ₂ (15). TGF [symbol] β₁-α₂M* and TGF- [symbol] ₂- [symbol] ₂M* complexes are cleared from plasma by the [symbol] liver (30). To test the possibility that fatty acids may be able to affect the plasma clearance of TGF [symbol] β and α₂M* complexes, ¹²⁵I-TGF [symbol] β₁ or ¹²⁵I-TGF- [symbol] ₂ were prei presence or absence of 10 μM arachidonic acid at room temperature for 30 min, and then injected into mice via tail vein according to published procedures (19). At several time intervals (10 sec, 1, 2, 3, 5, 10, 15, 20, 30 and 60 min) about 50 μl of blood was collected and counted by a γ-counter. As shown in FIGS. 7A and B, the estimated plasma clearance half times (t_{1/2}s) of free ¹²⁵I-TGF [symbol] β₁ (FIG. 7A) and ¹²⁵I-TGF [symbol] β₂ (FIG. 7B) were approximately 1-2 min. The t ₁+α₂M* or ¹²⁵I-TGF- [symbol] ₂+α₂M* were approximately 4 min. These t_{with published values of free} ¹²⁵I-TGF- [symbol] _{1,2} and ¹²⁵I-TGF- [symbol] _{1,2}-α₂M* complexes, respe (19). In the presence of arachidonic acid, the t_{1/2}s of ¹²⁵I-TGF [symbol] β₁+α₂M* and ¹²⁵I-TGF α₂M* were decreased to approximately 1-2 min; these are essentially identical to the t_{1/2}s of free ¹²⁵I-TGF- [symbol] ₁ and ¹²⁵I-TGF [symbol] β₂ (FIGS. 7A and B). In control experiments, arachidonic acid did no affect the plasma clearance of free ¹²⁵I-TGF [symbol] β₁ and ¹²⁵I-TGF- [symbol] ₂. These results suggest that arachidonic acid is capable of affecting the plasma clearance of TGF- [symbol] + [symbol] ₂M* by blocking complex formation.

### EXAMPLE 7

### Materials and Procedures

Materials—

Na¹²⁵I (17.4 Ci/mg), [5,6,8,9,11,12,14,15-³H] arachidonic acid (683 mCi/mg), [methyl-³H] thymidine (102 mCi/mg), chelate—Sepharose FF and Sephacryl® S-300 HR were purchased from Amersham Pharmacia Biotech (UK). TGF [symbol] β₁, TGF- [symbol] ₂ and TGF- [symbol] ₃ were obtained from Austral Biologicals (San Ramon, Calif.) and R&D Systems, Inc. (Minneapolis, Minn.). Disuccinimidyl suberate (DSS) was obtained from Pierce. Fatty acids (cis), fatty acid-derivatives and analogues and bovine serum albumin (A-7030) were purchased from Sigma Chemical Co. (St. Louis, Mo.). Mink lung epithelial cells (Mv1Lu) were grown and maintained in Dulbecco's modified Engle's medium (DMEM) containing 10% fetal calf serum (FCS). ICR mice were obtained from the Laboratory Animal Center, National Taiwan University College of Medicine, Taipei, Taiwan.

Preparation of Human α₂M and [symbol] ₂M*—Human [symbol] ₂M was purified from pooled citrate-treate human plasma using Zn²⁺ chelate—Sepharose® FF affinity chromatography followed by gel-filtration on Sephacryl® S-300 HR as described previously (20,21). [symbol] ₂M ( [symbol] ₂M*) activat by methylamine and plasmin were prepared as described previously (12,22).

Iodination of TGF [symbol] β-TGF- [symbol] ₁, TGF- [symbol] ₂ and TGF- [symbol] ₃ (5 μg) were each iod mCi of Na¹²⁵I using chloramine T according to the procedure of Huang et al. (12). The specific radioactivity of ¹²⁵I-TGF- [symbol] ₁, ¹²⁵I-TGF- [symbol] ₂ and ¹²⁵I-TGF- [symbol] ₁ was 1

Complex formation of ¹²⁵I-TGF-β and [symbol] ₂M*—The reaction mixture contained 10 μg of _{.} α₂M*, ˜1 nM of ¹²⁵I-TGF- [symbol] ₁, ¹²⁵I-TGF [symbol] β₂ or ¹²⁵I-TGF [symbol] acids (dissolved in 100% ethanol) in 0.05 ml of 50 mM HEPES-NaOH buffer, pH 7.4. The final concentration of ethanol in the reaction mixture was 0.5%. These fatty acids and fatty acid derivatives were soluble under the experimental conditions. After 30 min at room temperature, the complex formation of ¹²⁵I-TGF- [symbol] and_{.} α₂M* was determined by 5% non-denaturing polyacrylamide gel electrophoresis (PAGE) or by 7.5% SDS-PAGE following cross-linking by 0.6 mM DSS. After electrophoresis, the gel was stained with Coomassie blue and analyzed by autoradiography. The ¹²⁵I-TGF- [symbol] - [symbol] ₂M* complex which co-migrated with free [symbol] ₂M* was quantified using a PhosphoImager (Fuji).

Gel Filtration of ³H-arachidonic acid-α₂M* Complexes—The reaction mixture contained 100 μM ³H-arachidonic acid with or without 10 μg of [symbol] ₂M*, which was activated by methylamine and plasmin as described previously (12,22), or native [symbol] ₂M in 0.05 ml of 50 mM HEPES-NaOH buffer, pH 7.4. After 30 min at room temperature, the reaction mixtures were applied onto a column (0.7×40 cm) of Sephacryl® S-300 HR pre-equilibrated with 50 mM sodium phosphate buffer, 150 mM NaCl, pH 7.0. The column was then eluted with the same phosphate buffer and the fractional volume was ˜1 ml, 20 μl of which was counted with a scintillation counter and an another 20 μl of which was analyzed by SDS-PAGE followed by Coomassie blue staining (to locate fractions containing_{.} α₂M* or native [symbol] ₂M). The ³H-arachidonic acid—_{.} α₂M* complex co-chromatographed with [symbol] ₂M* or native_{.} α₂M. _{.} α₂M* whether activated by methylamine or plasmin, did not show significant differences in ability to bind ³H-arachidonic acid with respect to the stoichiometry of ³H-arachidonic acid and α₂M* in the complex.

Binding of ¹²⁵I-TGF- [symbol] ₂ to Mv1Lu cells—Mv1Lu cells grown on 24-well clustered dishes were incubated with various concentrations (1.25, 2.5, 5 and 10 pM) of ¹²⁵I-TGF [symbol] ₂ and _{. 2}M* (0 and 200 μg/ml) in the presence and absence of 30 μM arachidonic acid and 10 μM TGF [symbol] pep (19) in binding buffer (23). After 2.5 hr at 0° C., the cells were washed with binding buffer, and the cell-associated radioactivity was determined. All experiments were carried out in quadruplicate.

[Methyl-³H]-Thymidine Incorporation Assay—Mv1Lu cells were plated at a cell density of 7.5×10⁴ cells/well in DMEM containing 0.1% fetal calf serum in 48-well cluster dishes. After 4 hr at 37° C. (to allow cell adherence), cells were treated with various concentrations of TGF- [symbol] _{2,2}M* (0 or 200 μg/ml) and arachidonic acid (0, 0.1 or 1 μM). After 1h at 37° C., cells were pulsed with 1 μCi/ml [methyl-³H]-thymidine for 2 hr. The [methyl-³H]-th incorporation into cellular DNA was carried out in triplicate as described previously (23).

Luciferase Assay—Mv1Lu cells which had been plated on 12-well clustered dishes at a cell density of approximately 0.8-1.0×10⁵ cells/plate were transfected with 4-6 μg of p3TP-Lux using the calcium phosphate method (24). After 12 hr, the transfected cells were washed with phosphate buffered saline and allowed to grow in a medium containing 10% fetal calf serum for 12 hr. The medium was changed to DMEM with low serum concentration (0.2% fetal calf serum) and the cells were incubated for 4-6 hr. The cells were then treated for 20 hr with TGF-_{.}β₂ (0, 50 or 100 pM), α₂M* (0 or 200 μg/ml) and arachidonic acid (0, 12.5 or 25 μM) in the same low-serum medium. The cells were harvested and assayed for luciferase activity using the Promega kit according to the manufacturer's protocol. The luciferase activity was assayed in triplicate cell cultures and measured as arbitrary units (A.U.).

Plasma clearance of ¹²⁵I-TGF [symbol] β in the presence and absence of [symbol] ₂M*-¹²⁵I-TGF- [symbol] _{1 nM) or} ¹²⁵I-TGF- [symbol] ₂ (1 nM) was pre-incubated with [symbol] ₂M* (10 μg/50 μl) in prese of 10 μM arachidonic acid at room temperature for 30 min prior to injection into the lateral tail veins of mice anesthetized with ketamine as described previously (19). Blood samples (25 μL) were taken at 10 s, 1 min, 2, 3, 5, 10, 15, 20, 30 and 60 min from the retro-orbital venous plexus using heparinized hematocrit tubes. The radioactivity in the blood sample obtained at 10 s was taken as 100%.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of references herein is intended merely to summarize the assertions made by their authors and no admission is made that any reference constitutes prior art. Applicants reserve the right to challenge the accuracy and pertinence of the cited references.

What is claimed is:

1. A method for modulating the activity of a growth factor in a sample, which contains an activated α2-macroglobulin, comprising (a) contacting the sample with a fatty acid in an amount sufficient to inhibit the formation of a complex between the growth factor and the activated α2-macroglobulin, wherein (b) the fatty acid binds to the activated α2-macroglobulin.

2. The method of claim 1 wherein the fatty acid has a carbon chain length of at least 14.

3. The method of claim 2 wherein the fatty acid is a saturated fatty acid.

4. The method of claim 3 wherein the fatty acid is selected from the group consisting of myristic acid, palmitic acid and stearic acid.

5. The method of claim 4 wherein the fatty acid is myristic acid.

6. The method of claim 2 wherein the fatty acid is an unsaturated fatty acid.

7. The method of claim 6 wherein the fatty acid is selected from the group consisting of arachidonic acid, oleic acid, γ-linolenic acid, linoleic acid, palmitoleic acid and linolenic acid.

8. The method of claim 7 wherein the fatty acid is arachidonic acid.

9. The method of claim 1 wherein the growth factor is selected from the group consisting of platelet-derived growth factor-AA, platelet-derived growth factor-BB, vascular endothelial cell growth factor, fibroblast growth factors, interleukins, growth hormone, insulin, insulin-like growth factor-1, insulin-like growth factor-2, nerve growth factor, neurotrophins and TGF-β.

10. The method of claim 9 wherein the growth factor is TGF-β.

11. The method of claim 10 wherein the TGF-β is selected from the group consisting of TGF-β1, TGF-β2 and TGF-β3.

12. The method of claim 11 wherein the TGF-β is TGF-β1.

13. The method of claim 1 wherein the sample is a tissue or plasma.

14. The method of claim 13 wherein the tissue or plasma is in an animal.

15. The method of claim 14 wherein the animal is a mouse.

16. The method of claim 10 wherein the TGF-β activity in the sample is increased relative to the TGF-β activity in another sample to which no fatty acid is added.

17. The method of claim 10 wherein the formation of a complex between the TGF-β and the activated α2-macroglobulin is inhibited at least 10% relative to the formation of a complex between a TGF-β and an activated α2-macroglobulin in a sample to which no fatty acid is added.

18. The method of claim 10 wherein the formation of a complex between the TGF-β and the activated α2-macroglobulin is inhibited at least 20% relative to the formation of a complex between a TGF-β and an activated α2-macroglobulin in a sample to which no fatty acid is added.

19. The method of claim 10 wherein the formation of a complex between the TGF-β and the activated α2-macroglobulin is inhibited at least 40% relative to the formation of a complex between a TGF-β and an activated α2-macroglobulin in a sample to which no fatty acid is added.

20. The method of claim 10 wherein the formation of a complex between the TGF-β and the activated α2-macroglobulin is inhibited at least 60% relative to the formation of a complex between a TGF-β and an activated α2-macroglobulin in a sample to which no fatty acid is added.

21. The method of claim 10 wherein the formation of a complex between the TGF-β and the activated α2-macroglobulin is inhibited at least 80% relative to the formation of a complex between a TGF-β and an activated α2-macroglobulin in a sample to which no fatty acid is added.

22. A method for modulating the activity of a growth factor in a sample, which contains an α2-macroglobulin-growth factor complex, comprising (a) contacting the sample with a fatty acid in an amount sufficient to promote the dissociation of the α2-macroglobulin-growth factor complex, wherein (b) the fatty acid binds to the α2-macroglobulin portion of the α2-macroglobulin-growth factor complex and (c) the growth factor dissociates from α2-macroglobulin.

23. The method of claim 22 wherein the fatty acid has a carbon chain length of at least 14.

24. The method of claim 23 wherein the fatty acid is a saturated fatty acid.

25. The method of claim 24 wherein the fatty acid is selected from the group consisting of myristic acid, palmitic acid and stearic acid.

26. The method of claim 25 wherein the fatty acid is myristic acid.

27. The method of claim 23 wherein the fatty acid is an unsaturated fatty acid.

28. The method of claim 27 wherein the fatty acid is selected from the group consisting of arachidonic acid, oleic acid, γ-linolenic acid, linoleic acid, palmitoleic acid and linolenic acid.

29. The method of claim 28 wherein the fatty acid is arachidonic acid.

30. The method of claim 1 wherein the growth factor is selected from the group consisting of platelet-derived growth factor-AA, platelet-derived growth factor-BB, vascular endothelial cell growth factor, fibroblast growth factors, interleukins, growth hormone, insulin, insulin-like growth factor-1, insulin-like growth factor-2, nerve growth factor, neurotrophins and TGF-β.

31. The method of claim 30 wherein the growth factor is TGF-β.

32. The method of claim 31 wherein the TGF-β is selected from the group consisting of TGF-β1, TGF-β2 and TGF-β3.

33. The method of claim 32 wherein the TGF-β is TGF-β₁.

34. The method of claim 22 wherein the sample is a tissue or plasma.

35. The method of claim 34 wherein the tissue or plasma is in an animal.

36. The method of claim 35 wherein the animal is a mouse.

37. A method of blocking the inhibitory effects of activated α₂-macroglobulin on TGF-β activity or reversing the inhibitory effects of activated α₂-macroglobulin on TGF-β activity comprising (a) contacting a sample, which comprises an activated α₂-macroglobulin or an α₂-macroglobulin-TGF-β complex, with a fatty acid in an amount sufficient to (i) inhibit the formation of a complex between the TGF-β and the activated α₂-macroglobulin or (ii) promote the dissociation of the α₂-macroglobulin-TGF-β complex, wherein (b) the fatty acid binds to the activated α₂-macroglobulin or the α₂-macroglobulin portion of the α₂-macroglobulin-TGF-β complex.
